(19) United States
(12) Patent Application Publication
(10) Pub. No.: US 2022/0142825 A1
(43) Pub. Date: May 12, 2022

(54) TAMPON AND PAD BODY HAVING A FOAM ABSORBENT BODY

(71) Applicant: CHEN CHI HSIANG INDUSTRY LIMITED, Taichung City (TW)

(72) Inventor: Yu-Hsiang LIU, Taichung City (TW)

(73) Assignee: CHEN CHI HSIANG INDUSTRY LIMITED, Taichung City (TW)

(21) Appl. No.: 17/096,563

(22) Filed: Nov. 12, 2020

(51) Int. Cl. A61F 13/20; A61F 13/26; A61F 13/34

(52) U.S. Cl. CPC A61F 13/2025; A61F 13/202; A61F 13/2057; A61F 13/34; A61F 13/2071; A61F 13/266

(57) ABSTRACT

A pad body integrally molded from a thermoplastic resin foam material with high compressibility includes a foam absorbent body, an outer film layer formed on an outer portion of the foam absorbent body, and a pull string connected to the outer film layer. A tampon is also disclosed, and includes a tubular sleeve unit and the pad body. The tubular sleeve unit includes an outer tube and a push rod. The foam absorbent body and the outer film layer constitute an absorption section of the pad body. The absorption section is constrained by the outer tube in a compressed state, and is transformed to an expanded state when pushed by the push rod to move out of the receiving hole via the exit opening.

### FIELD

The disclosure relates to a hygiene product, more particularly to a tampon and a pad body having a foam absorbent body.

### BACKGROUND

A tampon, as disclosed in Taiwanese Patent No. M593832, includes a support sleeve and an absorbent cotton body disposed in the support sleeve. The support sleeve is a tubular object made after compressing and processing a cotton material, and has an open upper end and a closed lower end. A pull string is provided on the lower end of the support sleeve. The support sleeve is covered with a flexible and ductile film. A plurality of push-pull protrusions are integrally provided around an outer wall of the film.

In use, the tampon is inserted into the vagina. The tampon can be smoothly pushed from the vaginal opening into the vagina through the support sleeve, and menstrual blood can be absorbed through the strong absorption of the absorbent cotton body.

Although this kind of tampon can achieve its intended purpose, because the tampon is mostly made of cotton, there are many complicated processes for making fibers from raw materials and from the fibers to the tampon, so that the manufacturing cost of this kind of tampon is high.

### SUMMARY

Therefore, an object of the present disclosure is to provide a pad body having a foam absorbent body that is capable of overcoming the aforesaid drawback of the prior art.

According to this disclosure, a pad body is integrally molded from a thermoplastic resin foam material with high compressibility, and includes foam absorbent body, an outer film layer formed on an outer portion of the foam absorbent body, and a pull string connected to the outer film layer. The foam absorbent body and the outer film layer constitute an absorption section of the pad body. The outer film layer has a plurality of absorption holes communicating the foam absorbent body with the outside. The absorption section is transformable from a compressed state to an expanded state, and has a connecting end portion connected to the pull string, a top end portion opposite to the connecting end portion, and an intermediate portion between the connecting end portion and the top end portion.

Another object of the present disclosure is to provide a tampon having the pad body that is capable of overcoming the aforesaid drawback of the prior art.

According to this disclosure, a tampon includes a tubular sleeve unit and a pad body. The tubular sleeve unit includes an outer tube that defines a receiving hole, and a push rod movably disposed in the receiving hole. The outer tube has a tubular main portion, a head portion connected to the tubular main portion, and a bottom portion opposite to the head section. The head portion has a hemispherical shape, and has an exit opening communicating the receiving hole with the outside, and a plurality of angularly spaced-apart claw pieces connected to a top periphery of the tubular main portion, and surrounding the exit opening. Each claw piece has a connecting end connected to the top periphery of the tubular main portion, and a free end opposite to the connecting end. The claw pieces are movable between a closed state, in which the free ends of the claw pieces are close to each other, and an open state, in which the free ends of the claw pieces are moved away from each other.

The pad body is integrally molded from a thermoplastic resin foam material with high compressibility. The pad body includes a foam absorbent body, an outer film layer formed on an outer portion of the foam absorbent body, and a pull string connected to the outer film layer. The foam absorbent body and the outer film layer constitute an absorption section of the pad body. The absorption section is removably disposed in the receiving hole of the outer tube. The outer film layer has a plurality of absorption holes communicating the foam absorbent body with the outside. The absorption section is transformable from a compressed state to an expanded state. The absorption section is constrained by the outer tube in the compressed state, and is transformed from the compressed state to the expanded state when pushed by the push rod to move out of the receiving hole via the exit opening. The absorption section has a connecting end portion connected to the pull string, a top end portion opposite to the connecting end portion, and an intermediate portion between the connecting end portion and the top end portion.

### BRIEF DESCRIPTION OF THE DRAWINGS

Other features and advantages of the present disclosure will become apparent in the following detailed description of the embodiment with reference to the accompanying drawings, of which:

FIG. 1 is a perspective view of a tampon according to an embodiment of the present disclosure;

FIG. 2 is a sectional view of the embodiment, illustrating an absorption section of a pad body in a compressed state;

FIG. 3 illustrates the absorption section of the pad body of the embodiment in an expanded state;

FIG. 4 illustrates the embodiment in a state of use; and

FIG. 5 illustrates an alternative form of the absorption section of the pad body of the embodiment in the expanded state.

### DETAILED DESCRIPTION

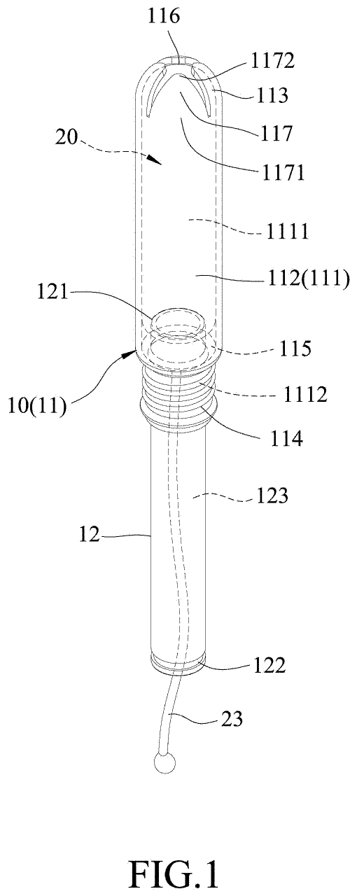

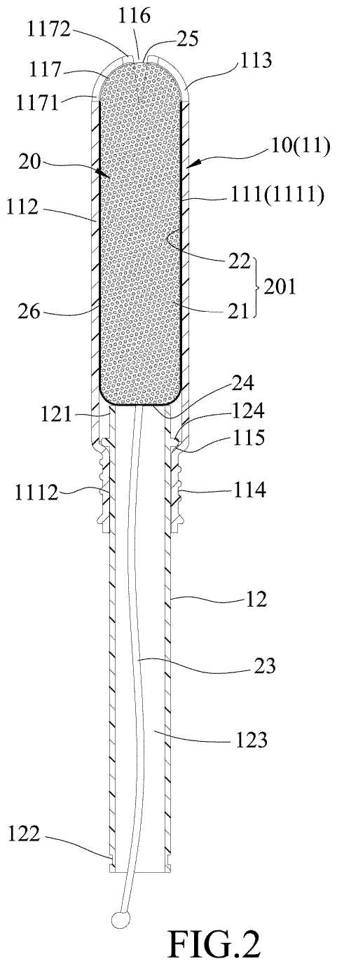

Referring to FIGS. 1 and 2, a tampon 100 according to an embodiment of the present disclosure is shown to include a tubular sleeve unit 10 and a pad body 20.

The tubular sleeve unit 10 includes an outer tube 11 defining a receiving hole 111, and a push rod 12 movably disposed in the receiving hole 111. The outer tube 11 is made of a soft plastic material that is easy to deform, and has a tubular main portion 112, a head portion 113 connected to the tubular main portion 112, and a bottom portion 114 opposite to the head section 113. The receiving hole 111 has a large diameter hole section 1111 defined by the tubular main portion 112, a small diameter hole section 1112 defined by the bottom portion 114, and a shoulder 115 between the large and small diameter hole portions 1111, 1112. The head portion 113 has a hemispherical shape, and has an exit opening 116 communicating the receiving hole 111 with the outside, and a plurality of angularly spaced-apart claw pieces 117 connected to a top periphery of the tubular main portion 112 and surrounding the exit opening 116. The claw pieces 117 have elasticity when stretched. Each claw piece 117 has a connecting end 1171 connected to the top periphery of the tubular main portion 112, and a free end 1172 opposite to the connecting end 1171. The claw pieces 117 are movable between a closed state (see FIG. 2), in which the free ends 1172 of the claw pieces 117 are close to each other, and an open state (not shown), in which the free ends 1172 of the claw pieces 117 are moved away from each other.

The push rod 12 is hollow, and has an inner end portion 121 extending into the receiving hole 111, an operating end portion 122 opposite to the inner end portion 121, a through hole 123 extending from the inner end portion 121 to the operating end portion 122, and an annular flange 124 provided on an outer periphery thereof in proximity to the inner end portion 121 and abuttable against the shoulder portion 115 within the receiving hole 111.

The pad body 20 is a one-piece body molded from a thermoplastic resin foam material with high compressibility. The thermoplastic resin foam material is selected from at least one of polyurethane (PU), polyethylene (PE), polypropylene (PP), and polyethylene terephthalate (PET). The pad body 20 includes a foam absorbent body 21, an outer film layer 22 formed on an outer portion of the foam absorbent body 21, and a pull string, 23 connected to the outer film layer 22. The outer film layer 22 covers a majority of the foam absorbent body 21, but not a top portion thereof. The outer film layer 22 has a plurality of absorption holes 221 communicating the foam absorbent body 21 with the outside. The pull string 23 has a density and strength greater than that of the foam absorbent body 21.

The foam absorbent body 21 and the outer film layer 22 constitute an absorption section 201 of the pad body 20. The absorption section 201 is removably disposed in the receiving hole 111, and has a connecting end portion 24 connected to the pull string 23, a top end portion 25 opposite to the connecting end portion 24, and an intermediate portion 26 between the connecting end portion 24 and the top end portion 25.

With reference to FIGS. 1 and 2, before the tampon 100 is used, the absorption section 201 of the pad body 20 is constrained by the outer tube 11 in a compressed state, and the pull string 23 extends out of the through hole 123 of the push rod 12. At this time, the claw pieces 117 are in the closed state, that is, the free ends 1172 thereof are close to each other.

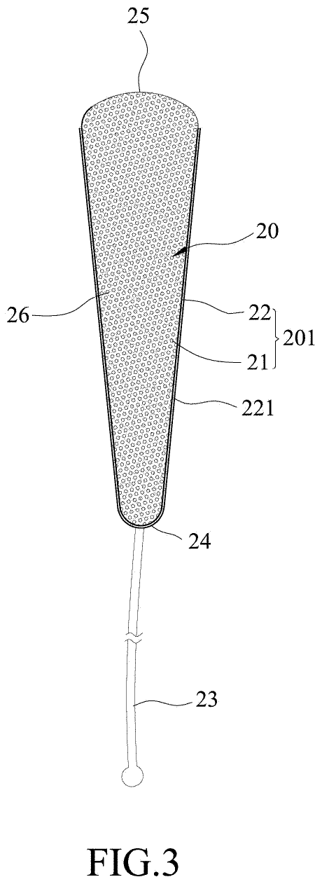

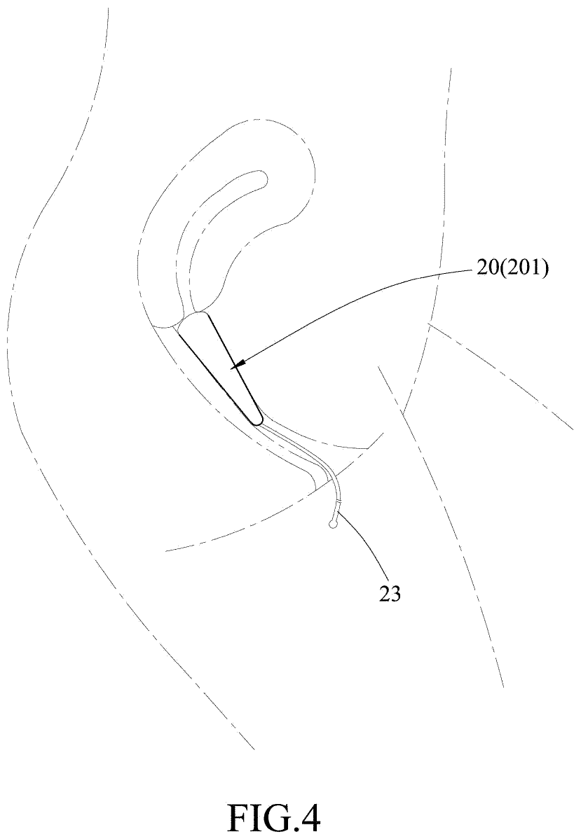

When the head portion 113 of the outer tube 11 of the tampon 100 is inserted into the vagina of a user, the operating end portion 122 of the push rod 12 is pushed to move the inner end portion 121 of the push rod 12 toward the head portion 113 of the outer tube 11. The inner end portion 121 of the push rod 12, in turn, pushes the absorption section 201 of the pad body 20 to gradually move out of the receiving hole 111 via the exit opening 116. As the absorption section 201 moves out of the receiving hole 111, the absorption section 201 pushes the free ends 1172 of the claw pieces 117 to move away from each other to the open state, so that it can move out of the receiving hole 111 into the vagina of the user, as shown in FIG. 4. At this time, a portion of the pull string 23 is located outside of a body of the user. Since the absorption section 201 is not constrained by the outer tube 11 when it is located in the vagina of the user, the absorption section 201 can automatically expand and transform into an expanded state (see FIG. 3). In this embodiment, the absorption section 201 has a conical shape in the expanded state, and has a width that gradually decreases from the top end portion 25 to the connecting end portion 24. Menstrual blood is absorbed through the top end portion 25 of the absorption section 201.

To remove the absorption section 201 of the pad body 20 from the vagina, the portion of the pull string 42 located outside of the body of the user is pulled, and through the conical shape of the absorption section 201, the absorption section 201 can be smoothly removed from the vagina of the user.

Hence, use of the tampon 100 of this disclosure is very easy. Further, because the foam absorbent body 21, the outer film layer 22 and the pull string 23 of the pad body 20 are integrally formed as one piece, the manufacture of the tampon 100 is also very convenient, so that the manufacturing processes thereof can be simplified and the manufacturing costs thereof can be reduced. It is worth to mention herein that the foam absorbent body 21, the outer film layer 22 and the pull string 23 of the pad body 20 can be formed directly inside a mold (not shown) and the outer film layer 22 is formed naturally as a film during molding. Through the provision of the outer film layer 22, the absorption section 201 can be smoothly inserted into or removed from the vagina of the user.

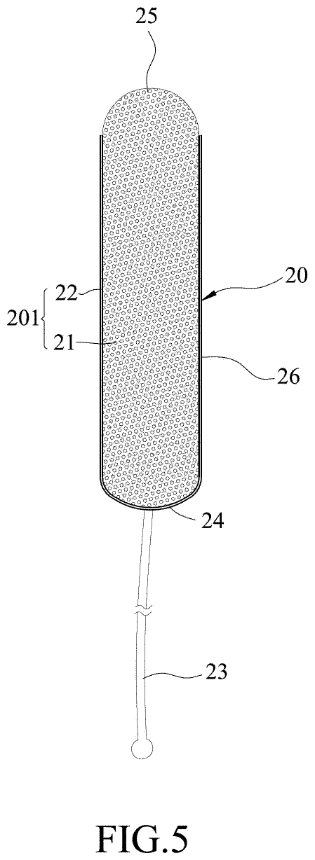

FIG. 5 illustrates an alternative form of the absorption section 201′ of the pad body 20′. In this case, the absorption section 201′ of the pad body 20′ has a cylindrical shape in the expanded state, and has a uniform width from the connecting end portion 24 to the top end portion 25.

In summary, the pad body 20, 20′ of this disclosure is integrally molded from a thermoplastic resin foam material with high compressibility, and the foam absorbent body 21, the outer film layer 22 and the pull string 23 of the pad body 20, 20′ are integrally formed as one piece, so that the manufacture of the tampon 100 of this disclosure s easy, thereby reducing the manufacturing costs thereof.

While the present disclosure has been described in connection with what is considered the most practical embodiments, it is understood that this disclosure is not limited to the disclosed embodiment but is intended to cover various arrangements included within the spirit and scope of the broadest interpretation so as to encompass all such modifications and equivalent arrangements.

What is claimed is:

1. A pad body integrally molded from a thermoplastic resin foam material with high compressibility and comprising a foam absorbent body, an outer film layer formed on an outer portion of said foam absorbent body, and a pull string connected to said outer film layer, said foam absorbent body and said outer film layer constituting an absorption section of said pad body, said outer film layer having a plurality of absorption holes communicating said foam absorbent body with the outside, said absorption section being transformable from a compressed state to an expanded state, said absorption section having a connecting end portion connected to said pull string, a top end portion opposite to said connecting end portion, and an intermediate portion between said connecting end portion and said top end portion.

2. The pad body as claimed in claim 1, wherein said outer film layer covers a majority of said foam absorbent body, but not a top portion thereof.

3. The pad body as claimed in claim 1, wherein said absorption section has a conical shape in the expanded state, and has a width that gradually decreases from said top end portion to said connecting end portion.

4. The pad body as claimed in claim 1, wherein said absorption section has a cylindrical shape in the expanded state.

5. The pad body as claimed in claim 1, wherein the thermoplastic resin foam material is selected from, at least one of polyurethane, polyethylene, polypropylene, and polyethylene terephthalate.

6. A tampon comprising:
a tubular sleeve unit including an outer tube that defines a receiving hole, and a push rod movably disposed in said receiving hole, said outer tube having a tubular main portion, a head portion connected to said tubular main portion, and a bottom portion opposite to said head section, said head portion having a hemispherical shape and having an exit opening communicating said receiving hole with the outside, and plurality of angularly spaced-apart claw pieces connected to a top periphery of said tubular main portion and surrounding said exit opening, each of said claw pieces having a connecting end connected to said top periphery of said tubular main portion, and a free end opposite to said connecting end, said claw pieces being movable between a closed state, in which said free ends of said claw pieces are close to each other, and an open state, in which said free ends of said claw pieces are moved away from each other; and
a pad body integrally molded from a thermoplastic resin foam material with high compressibility and including a foam absorbent body, an outer film layer formed on an outer portion of said foam absorbent body, and a pull string connected to said outer film layer, said foam absorbent body and said outer film layer constituting an absorption section of said pad body, said absorption section being removably disposed in said receiving hole of said outer tube, said outer film layer having a plurality of absorption holes communicating said foam absorbent body with the outside, said absorption section being transformable from a compressed state to an expanded state, said absorption section being constrained by said outer tube in the compressed state, and being transformed from the compressed state to the expanded state when pushed by said push rod to move out of said receiving hole via said exit opening, said absorption section having a connecting end portion connected to said pull string, a top end portion opposite to said connecting end portion, and an intermediate portion between said connecting end portion and said top end portion.

7. The tampon as claimed in claim 6, wherein said outer film layer covers a majority of said foam absorbent body, but not a top portion thereof.

8. The tampon as claimed in claim 6, wherein said absorption section has a conical shape in the expanded state, and has a width that gradually decreases from said top end portion to said connecting end portion.

9. The tampon as claimed in claim 6, wherein said absorption section has a cylindrical shape in the expanded state.

10. The tampon as claimed in claim 6, wherein the thermoplastic resin foam material is selected from at least one of polyurethane, polyethylene, polypropylene, and polyethylene terephthalate.
